(19) United States
(12) Patent Application Publication
(10) Pub. No.: US 2007/0149632 A1
(43) Pub. Date: Jun. 28, 2007

(54) Short chain polyethers for rigid polyurethane foams

(76) Inventors: Karl W. Haider, Wexford, PA (US); Keith J. Headley, Paden City, WV (US); Don S. Wardius, Pittsburgh, PA (US)

Correspondence Address: BAYER MATERIAL SCIENCE LLC, 100 BAYER ROAD, PITTSBURGH, PA, 15205, US

(21) Appl. No.: 11/315,531

(22) Filed: Dec. 22, 2005

(51) Int. Cl. C08G 18/00; C08F 20/00; C07C 43/11

(52) U.S. Cl. 521/172; 525/438; 568/679

(57) ABSTRACT

The present invention provides a short-chain polyether polyol having a number average molecular weight of less than about 1,200 g/mole and produced by alkoxylating an initiator in the presence of a basic catalyst having at least one cation chelated with about 0.5 wt. % to about 20 wt. % of a polyoxyethylene-containing compound, wherein the weight percentages are based on the weight of the short-chain polyether polyol. The inventive short-chain polyols may be used to produce rigid polyurethane foams and non-cellular polyurethanes.

### FIELD OF THE INVENTION

The present invention relates in general to polyether polyols, and more specifically, to a short-chain polyether polyol having a molecular weight of less than about 1,200 g/mole and produced by alkoxylating an initiator in the presence of a basic catalyst having at least one cation thereof chelated with from about 0.5 wt. % to about 20 wt. % of a polyoxyethylene-containing compound.

### BACKGROUND OF THE INVENTION

It has been known for many years that cyclic ethers complex potassium ions strongly. Crown ethers were discovered in the 1960's by Charles Pederson and in 1987 he was awarded the Nobel Prize for his efforts. The ability of cyclic ethers to strongly complex metal ions has led to much scientific work. Unfortunately, because crown ethers are difficult to make, expensive and highly toxic, they have never found wide commercial application. Perhaps, because crown ethers were discovered first, many in the art have overlooked the strong complexing abilities possessed by non-cyclic polyethers. Among the advantages are the ready availability, low cost and the fact that polymers and oligomers of ethylene oxide are so non-toxic as to be acceptable for use as food additives.

Although the concept of using polyethylene glycols (“PEGs”) for rate enhancement of the KOH-catalyzed alkoxylation of long-chain polyols is known in the art (See “Synthesis of Polyether Polyols for Flexible Polyurethane Foams with Complexed Counter-Ion” by Mihail Ionescu, Viorica Zugravu, Ioana Mihalache and Ion Vasile, Cellular Polymers IV. International Conference. 4th, Shrewsbury, UK, Jun. 5-6, 1997 Paper 8, 1-8. Editor(s): Buist, J. M.), there are no published reports of extending this concept to short-chain polyol syntheses.

A commonly-assigned U.S. patent application filed on an even date herewith and entitled “Base-catalyzed alkoxylation in the presence of polyoxyethylene-containing compounds”, (Atty. Docket No. PO8708, U.S. Ser. No. ______) discloses a molecular weight dependency for a polyoxyethylene-containing additive which acts as a chelating agent in the base-catalyzed alkoxylation of long-chain polyethers.

A second commonly-assigned U.S. patent application also filed on an even date herewith and entitled “Base-catalyzed alkoxylation in the presence of non-linear polyoxyethylene-containing compounds”, (Atty. Docket No. PO8709, U.S. Ser. No. ______) discloses a non-linear, at least trifunctional polyoxyethylene-containing additive as a chelating agent for the base-catalyzed alkoxylation of long-chain polyethers, with no detrimental effect on flexible foams produced therefrom.

Finally, a third commonly-assigned U.S. patent application also filed on an even date herewith and entitled “Long-chain polyether polyols”, (Atty. Docket No. PO8706, U.S. Ser. No. ______) discloses a polyoxyethylene-containing initiator as a chelating agent in the alkoxylation of long-chain polyethers.

The starter mix for short chain polyols typically contains a mixture of polyhydroxyl or polyamino functional starters ranging in functionality from 2 to 8 (e.g., propylene glycol, glycerine, trimethylolpropane ethylene diamine, toluene diamine, sucrose, sorbitol), and often includes water. It was heretofore unknown what effect such PEGs would have on the base-catalyzed synthesis of short chain polyols, i.e., those with a molecular weight of less than about 1,200 g/mole, from these mixtures.

### SUMMARY OF THE INVENTION

Accordingly, the present invention obviates problems inherent in the art by providing a short-chain polyether polyol having a number average molecular weight of less than about 1,200 g/mole and produced by alkoxylating an initiator in the presence of a basic catalyst having at least one cation chelated with about 0.5 wt. % to about 20 wt. % of a polyoxyethylene-containing compound. The inventive short-chain polyols may be used to provide rigid polyurethane foams and non-cellular polyurethanes.

These and other advantages and benefits of the present invention will be apparent from the Detailed Description of the Invention herein below.

### DETAILED DESCRIPTION OF THE INVENTION

The present invention will now be described for purposes of illustration and not limitation. Except in the operating examples, or where otherwise indicated, all numbers expressing quantities, percentages, OH numbers, functionalities and so forth in the specification are to be understood as being modified in all instances by the term “about.” Equivalent weights and molecular weights given herein are number average equivalent weights and number average molecular weights respectively, unless indicated otherwise.

The present invention provides a short-chain polyether polyol having a number average molecular weight of less than 1,200 g/mole and produced by alkoxylating an initiator in the presence of a basic catalyst having at least one cation chelated with 0.5 wt. % to 20 wt. % of a polyoxyethylene-containing compound, wherein the weight percentages are based on the weight of the short-chain polyether polyol.

The present invention further provides a process for producing a short-chain polyether polyol involving alkoxylating an initiator in the presence of a basic catalyst having at least one cation chelated with 0.5 wt. % to 20 wt. % of a polyoxyethylene-containing compound, wherein the short-chain polyether polyol has a number average molecular weight of less than 1,200 g/mole, wherein the weight percentages are based on the weight of the short-chain polyether polyol.

The present invention still further provides a rigid polyurethane foam made from the reaction product of at least one polyisocyanate and at least one short chain polyether polyol having a number average molecular weight of less than 1,200 g/mole and produced by alkoxylating an initiator in the presence of a basic catalyst having at least one cation chelated with 0.5 wt. % to 20 wt. % of a polyoxyethylene-containing compound, optionally in the presence of at least one of blowing agents, surfactants, other cross-linking agents, extending agents, pigments, flame retardants, catalysts and fillers, wherein the weight percentages are based on the weight of the short-chain polyether polyol.

The present invention yet further provides a process for producing a rigid polyurethane foam involving reacting at least one polyisocyanate and at least one short chain polyether polyol having a number average molecular weight of less than 1,200 g/mole and produced by alkoxylating an initiator in the presence of a basic catalyst having at least one cation chelated with 0.5 wt. % to 20 wt. % of a polyoxyethylene-containing compound, optionally in the presence of at least one of blowing agents, surfactants, other cross-linking agents, extending agents, pigments, flame retardants, catalysts and fillers, wherein the weight percentages are based on the weight of the short-chain polyether polyol.

By “short-chain” polyether polyol, the inventors herein mean a polyether polyol having a number average molecular weight of less than 1,200 g/mole, preferably from 300 to 1,000 g/mole, more preferably from 500 to 900 g/mole. The molecular weight of the inventive polyols may be in an amount ranging between any combination of these values, inclusive of the recited values.

The short-chain polyether polyols of the present invention are made by basic catalysis, the general conditions of which are familiar to those skilled in the art. The basic catalyst may be any basic catalyst known in the art, more preferably the basic catalyst is one of potassium hydroxide, sodium hydroxide, barium hydroxide and cesium hydroxide; most preferably the basic catalyst is potassium hydroxide.

Suitable initiator compounds include, but are not limited to, C₁-C₃₀ monols, ethylene glycol, diethylene glycol, triethylene glycol, propylene glycol, dipropylene glycol, tripropylene glycol, neopentyl glycol, 1,3 propanediol, 1,4 butanediol, 1,2 butanediol, 1,3 butanediol, 2,3 butanediol, 1,6 hexanediol, water, glycerin, trimethylolpropane, trimethylolethane, ethylene diamine, mixture of isomers of toluene diamine, pentaerythritol, α-methylglucoside, sorbitol, mannitol, hydroxymethylglucoside, hydroxypropylglucoside, sucrose, N,N,N′,N′-tetrakis[2-hydroxyethyl or 2-hydroxypropyl]ethylene diamine, 1,4-cyclohexanediol, cyclohexanedimethanol, hydroquinone, resorcinol and the like. Nominal initiator functionality, which is understood to represent the ratio of the total number of equivalents of active hydrogens (as determined by the Zerewitinoff method) to moles in the starter mixture is from 1 to 8 or more, preferably from 3 to 6. The functionality of the initiators useful in the present invention may be in an amount ranging between any combination of these values, inclusive of the recited values. Any mixtures of monomeric initiators or their oxyalkylated oligomers may also be utilized. Preferred initiator compounds for short-chain polyether polyol of the present invention are mixtures of propylene glycol, sucrose, and water having functionality of 4-6.

The polyoxyethylene-containing compound, such as a polyethylene glycol, is added to chelate at least one of the cations of the basic catalyst during the alkoxylation in the inventive short-chain polyether polyol production process. The polyoxyethylene-containing compounds suitable in the present invention are understood to be ethoxylates of alcohols, diols, or polyols, such as a polyethylene glycol (PEG) or TPEG (available from Dow Chemical). This polyoxyethylene-containing compound preferably has a hydroxy functionality of 1-8 more preferably from 2 to 6 and most preferably from 2 to 3. Alternatively, the hydroxy functionality of the polyoxyethylene-containing compound may be capped with alkyl, preferably methyl, groups as is known to those skilled in the art. The functionality of the polyoxyethylene-containing compound may be in an amount ranging between any combination of these values, inclusive of the recited values. The polyoxyethylene-containing compound preferably has a molecular weight of from 150 to 1,200 more preferably from 200 to 1,000 and most preferably from 250 to 400. The polyoxyethylene-containing compound may have a molecular weight in an amount ranging between any combination of these values, inclusive of the recited values.

The polyoxyethylene-containing compound is preferably added in an amount of from 0.5 to 20 wt. %, more preferably from 1 to 10 wt. %, and most preferably in an amount of from 2 to 7 wt. %, wherein the weight percentages are based on the final weight of the short-chain polyether polyol. The polyoxyethylene-containing compound may be added in an amount ranging between any combination of these values, inclusive of the recited values.

The alkylene oxides useful in alkoxylating the initiator to produce the inventive short-chain polyether polyols include, but are not limited to, ethylene oxide, propylene oxide, oxetane, 1,2- and 2,3-butylene oxide, isobutylene oxide, epichlorohydrin, cyclohexene oxide, styrene oxide, and the higher alkylene oxides such as the C₅-C₃₀ α-alkylene oxides. Propylene oxide alone or mixtures of propylene oxide with ethylene oxide or another alkylene oxide are preferred. Other polymerizable monomers may be used as well, e.g. anhydrides and other monomers as disclosed in U.S. Pat. Nos. 3,404,109, 3,538,043 and 5,145,883, the contents of which are herein incorporated in their entireties by reference thereto.

The inventive short-chain polyether polyols may preferably be reacted with a polyisocyanate, optionally in the presence of blowing agents, surfactants, cross-linking agents, extending agents, pigments, flame retardants, catalysts and fillers to produce rigid polyurethane foams.

Suitable polyisocyanates are known to those skilled in the art and include unmodified isocyanates, modified polyisocyanates, and isocyanate prepolymers. Such organic polyisocyanates include aliphatic, cycloaliphatic, araliphatic, aromatic, and heterocyclic polyisocyanates of the type described, for example, by W. Siefken in Justus Liebigs Annalen der Chemie, .562, pages 75 to 136. Examples of such isocyanates include those represented by the formula
Q(NCO)ₙ
in which n is a number from 2-5, preferably 2-3, and Q is an aliphatic hydrocarbon group; a cycloaliphatic hydrocarbon group; an araliphatic hydrocarbon group; or an aromatic hydrocarbon group.

Examples of suitable isocyanates include ethylene diisocyanate; 1,4-tetramethylene diisocyanate; 1,6-hexamethylene diisocyanate; 1,12-dodecane diisocyanate; cyclobutane-1,3-diisocyanate; cyclohexane-1,3- and -1,4-diisocyanate, and mixtures of these isomers; 1-isocyanato-3,3,5-trimethyl-5-isocyanatomethylcyclohexane (isophorone diisocyanate;. German Auslegeschrift 1,202,785 and U.S. Pat. No. 3,401,190); 2,4- and 2,6-hexahydrotoluene diisocyanate and mixtures of these isomers; dicyclohexylmethane-4,4′-diisocyanate (hydrogenated MDI, or HMDI); 1,3-and 1,4-phenylene diisocyanate; 2,4- and 2,6-toluene diisocyanate and mixtures of these isomers (TDI); diphenylmethane-2,4′- and/or -4,4′-diisocyanate (MDI); polymeric diphenylmethane diisocyanate (PMDI), naphthylene-1,5-diisocyanate; triphenylmethane-4,4′,4″-triisocyanate; polyphenyl-polymethylene-polyisocyanates of the type which may be obtained by condensing aniline with formaldehyde, followed by phosgenation (crude MDI), which are described, for example, in GB 878,430 and GB 848,671; norbornane diisocyanates, such as described in U.S. Pat. No. 3,492,330; m- and p-isocyanatophenyl sulfonylisocyanates of the type described in U.S. Pat. No. 3,454,606; perchlorinated aryl polyisocyanates of the type described, for example, in U.S. Pat. No. 3,227,138; modified polyisocyanates containing carbodiimide groups of the type described in U.S. Pat. No. 3,152,162; modified polyisocyanates containing urethane groups of the type described, for example, in U.S. Pat. Nos. 3,394,164 and 3,644,457; modified polyisocyanates containing allophanate groups of the type described, for example, in GB 994,890, BE 761,616, and NL 7,102,524; modified polyisocyanates containing isocyanurate groups of the type described, for example, in U.S. Pat. No. 3,002,973, German Patentschriften 1,022,789, 1,222,067 and 1,027,394, and German Offenlegungsschriften 1,919,034 and 2,004,048; modified polyisocyanates containing urea groups of the type described in German Patentschrift 1,230,778; polyisocyanates containing biuret groups of the type described, for example, in German Patentschrift 1,101,394, U.S. Pat. Nos. 3,124,605 and 3,201,372, and in GB 889,050; polyisocyanates obtained by telomerization reactions of the type described, for example, in U.S. Pat. No. 3,654,106; polyisocyanates containing ester groups of the type described, for example, in GB 965,474 and GB 1,072,956, in U.S. Pat. No. 3,567,763, and in German Patentschrift 1,231,688; reaction products of the above-mentioned isocyanates with acetals as described in German Patentschrift 1,072,385; and polyisocyanates containing polymeric fatty acid groups of the type described in U.S. Pat. No. 3,455,883. It is also possible to use the isocyanate-containing distillation residues accumulating in the production of isocyanates on a commercial scale, optionally in solution in one or more of the polyisocyanates mentioned above. The polymeric diphenylmethane diisocyanates are particularly preferred. Those skilled in the art will recognize that it is also possible to use mixtures of the polyisocyanates described above.

Prepolymers may also be employed in the preparation of the inventive foams. Prepolymers may be prepared by reacting an excess of organic polyisocyanate or mixtures thereof with a minor amount of an active hydrogen-containing compound as determined by the well-known Zerewitinoff test, as described by Kohler in Journal of the American Chemical Society, 49, 3181(1927). These compounds and their methods of preparation are known to those skilled in the art. The use of any one specific active hydrogen compound is not critical; any such compound can be employed in the practice of the present invention.

Suitable additives optionally included in the rigid polyurethane foam forming formulations of the present invention include, for example, stabilizers, catalysts, cell regulators, reaction inhibitors, plasticizers, fillers, crosslinking or extending agents, blowing agents, etc.

Stabilizers which may be considered suitable for the inventive foam forming process include, for example, polyether siloxanes, and preferably those which are insoluble in water. Compounds such as these are generally of such a structure that a relatively short chain copolymer of ethylene oxide and propylene oxide is attached to a polydimethylsiloxane residue. Such stabilizers are described in, for example, U.S. Pat. Nos. 2,834,748, 2,917,480 and 3,629,308.

Catalysts suitable for the foam forming process of the present invention include those which are known in the art. These catalysts include, for example, tertiary amines, such as triethylamine, tributylamine, N-methylmorpholine, N-ethylmorpholine, N,N,N′,N′-tetramethylethylenediamine, pentamethyl-diethylenetriamine and higher homologues (as described in, for example, DE-A 2,624,527 and 2,624,528), 1,4-diazabicyclo(2.2.2)octane, N-methyl-N′-dimethyl-aminoethylpiperazine, bis-(dimethylaminoalkyl)piperazines, N,N-dimethylbenzylamine, N,N-dimethylcyclohexylamine, N,N-diethyl-benzylamine, bis-(N,N-diethylaminoethyl) adipate, N,N,N′,N′-tetramethyl-1,3-butanediamine, N,N-dimethyl-β-phenylethylamine, 1,2-dimethylimidazole, 2-methylimidazole, monocyclic and bicyclic amines together with bis-(dialkylamino)alkyl ethers, such as 2,2-bis-(dimethylaminoethyl) ether.

Other suitable catalysts which may be used in producing the inventive polyurethane foams include, for example, organometallic compounds, and particularly, organotin compounds. Organotin compounds which may be considered suitable include those organotin compounds containing sulfur. Such catalysts include, for example, di-n-octyltin mercaptide. Other types of suitable organotin catalysts include, preferably tin(II) salts of carboxylic acids such as, for example, tin(II) acetate, tin(II) octoate, tin(II) ethylhexoate and/or tin(II) laurate, and tin(IV) compounds such as, for example, dibutyltin oxide, dibutyltin dichloride, dibutyltin diacetate, dibutyltin dilaurate, dibutyltin maleate and/or dioctyltin diacetate.

Preferably auxiliary blowing agents (“ABAs”) are used in the foams made according to the present invention, although water, alone, or in combination with these ABAs can be used. The ABAs are well known in the art to produce rigid foams and include hydrocarbons, fluorocarbons, hydrofluorocarbons, hydrochlorocarbons, hydrochlorofluorcarbons, chlorofluorocarbons, and carbon dioxide. Suitable blowing agents include, but are not limited to, HCFC-141b (1-chloro-1,1-difluoroethane), HCFC-22 (monochlorodifluoromethane), HFC-245fa (1,1,1,3,3-pentafluoropropane), HFC-134a (1,1,1,2-tetrafluoroethane), HFC-365mfc (1,1,1,3,3-pentafluorobutane), cyclopentane, normal pentane, isopentane, LBL-2(2-chloropropane), trichlorofluoromethane, CCl₂ FCClF₂, CCl₂ FCHF₂, trifluorochloropropane, 1-fluoro-1,1-dichloroethane, 1,1,1-trifluoro-2,2-dichloroethane, methylene chloride, diethylether, isopropyl ether, methyl formate, carbon dioxide and mixtures thereof.

Where included, water functions as a blowing by reacting with the isocyanate component to chemically form carbon dioxide gas plus an amine moiety which reacts further with the polyisocyanate to form urea backbone groups.

### EXAMPLES

The present invention is further illustrated, but is not to be limited, by the following examples. All quantities given in “parts” and “percents” are understood to be by weight, unless otherwise indicated.

PEG-300, PEG-400, and PEG-600 are polyethylene glycols having number average molecular weights of 300, 400 and 600 g/mole, respectively, and are commercially available from Aldrich Chemical Company. TPEG-990 is an ethoxylated glycerine having a number average molecular weight of 990 g/mole, commercially available from Dow Chemical Company

### Examples 1-8

A sucrose/propylene glycol/water started polyether was prepared according to the following procedure using the amount of each component as specified in Table I (values in grams). Control experiments were performed without any polyoxyethylene-containing compounds (Examples C-1 and C-2). Examples 3-8 were prepared according to the invention and contained the indicated polyoxyethylene-containing compounds.

In all cases, the water, KOH solution, propylene glycol, sucrose, and PEG additive (for examples prepared according to the invention) were charged into a five-gallon polyether polyol reactor. The reactor was purged of oxygen by pressurizing to 40 psia with nitrogen,-evacuating to 20 psia and repeating three times. The vacuum valve to the reactor was closed, and the mixture was heated to 100° C. Nitrogen was added to the reactor until a pressure of 20 psia was reached. A propylene oxide (PO) feed into the reactor was initiated. The PO feed rate was controlled via a feedback loop to maintain a total reactor pressure of 45 psia. The grams of PO indicated in Table I as PO-1 were added and the feed was stopped and allowed to cook until the pressure stopped decreasing, indicating the PO was consumed. The time required for the PO addition was recorded. The vacuum valve to the reactor was opened and the reaction mixture was heated under full vacuum to de-water.

De-watering continued at 100° C. until the water level reached 1.95 to 2.0%, as determined by Karl-Fischer titration. Where necessary, water was added back into the reaction mixture to bring the water content into this range. The mixture was heated to 110° C., sufficient nitrogen was added to bring the reactor pressure to 20 psia, and the second PO feed (PO-2) was initiated. Over the first 120 minutes of the feed, the temperature was increased up to 120° C. in a linear fashion. Again, the PO feed rate was controlled via a feedback loop to maintain 45 psia of pressure during the feed. The time required for the second PO feed was recorded, and the total PO addition time determined by adding the time required for both PO feeds is shown in Table II. Sulfuric acid was added to neutralize the KOH, the product was filtered and characterized by viscosity at 25° C., hydroxyl number and appearance (turbid or not).

As can be appreciated by reference to Tables I and II below, in Examples 3 and 4, TPEG-990 (3%) was added to the reaction mixture and an equal number of equivalents of either sucrose (Ex. 3) or propylene glycol (Ex. 4) were removed. At the same KOH catalyst level as comparative example C-1 (0.3%), the propoxylation time was reduced from 15 to about 10 hours. Examples 5-8, where various polyoxyethylene-containing additive were added according to the invention and an equal number of equivalents of propylene glycol were removed, propoxylation time was reduced from the 9 hours of the control (Ex C-2; KOH=0.7%) to between 6 and 7.3 hours, at the same KOH level. This corresponds to feed time reductions on the order of 20-30% at 0.7 and 0.3% KOH levels, respectively.

Over the molecular weight range from 300-1,000 g/mole, there appeared to be very little dependence on the molecular weight of the polyoxyethylene-containing additive's rate accelerating effectiveness. However the lower molecular weight oxyethylene containing additive (PEG-300) yielded a non-turbid sample, whereas the higher molecular weight additives produced turbid samples in most cases.

TABLE I

| | Ex. C-1 | Ex. C-2 | Ex. 3 | Ex. 4 | Ex. 5 | Ex. 6 | Ex. 7 | Ex. 8 |
|---|---|---|---|---|---|---|---|---|
| Sucrose | 5,378 | 5,378 | 5,310 | 5,382 | 5,379 | 5,378 | 5,380 | 5,380 |
| Water | 1,076 | 1,077 | 1,078 | 1,076 | 1,078 | 1,077 | 1,078 | 1,076 |
| Propylene glycol | 432 | 432 | 438 | 370 | 369 | 364 | 364 | 296 |
| 45% KOH (0.3% final) | 125 | 283 | 125 | 125 | 284 | 283 | 279 | 283 |
| TPEG-990 | | | 541 | 537 | 541 | | | |
| PEG-600 | | | | | | 547 | | |
| PEG-400 | | | | | | | 540 | |
| PEG-300 | | | | | | | | 539 |
| PO-I feed | 5,980 | 5,980 | 5,980 | 5,980 | 5,980 | 5,980 | 5,980 | 5,980 |
| PO-II feed | 6,210 | 6,210 | 6,210 | 6,210 | 6,210 | 6,210 | 6,210 | 6,210 |
| Total PO feed | 12,190 | 12,190 | 12,190 | 12,190 | 12,190 | 12,190 | 12,190 | 12,190 |

TABLE II

| | Ex. C-1 | Ex. C-2 | Ex. 3 | Ex. 4 | Ex. 5 | Ex. 6 | Ex. 7 | Ex. 8 |
|---|---|---|---|---|---|---|---|---|
| Additive (% in product) | None | None | TPEG-990 | TPEG-990 | TPEG-990 | PEG-600 | PEG-400 | PEG-300 |
| | | | (3%) | (3%) | (3%) | (3%) | (3%) | (3%) |
| KOH (%) | 0.3 | 0.7 | 0.3 | 0.3 | 0.7 | 0.7 | 0.7 | 0.7 |
| Total feed (hours) | 15.0 | 9.0 | 10.25 | 10.0 | 7.0 | 6.0 | 7.3 | 6.5 |
| OH number (mg KOH/g) | 485.3 | 480 | 475.1 | 475.8 | 458.5 | 458 | 474 | 465 |
| Viscosity (cP)* | 32,942 | 32,238 | 27,868 | 37,865 | 28,863 | 27,328 | 28,228 | 28,804 |
| | | | (24,968) | (33,299) | (37,343) | | | |
| Neutralization | sulfuric | sulfuric | sulfuric | sulfuric | sulfuric | sulfuric | sulfuric | sulfuric |
| | acid | acid | acid | acid | acid | acid | acid | acid |
| Turbidity | no | no | yes | no | yes | yes | yes | no |

*values in parentheses represent corrected viscosities to 470 hydroxyl number using an empirically determined relationship between viscosity and hydroxyl number

### Examples 9-15

A sucrose/water-started polyether was prepared according to the following procedure using the amount of each component as specified in Table III (values in grams). Control experiments were performed without any polyoxyethylene-containing additive (Examples C-9, C-10 and C-11). Examples 12-15 were prepared according to the invention and contained the indicated polyoxyethylene-containing additive.

In all cases, the water, KOH solution, sucrose, and polyoxyethylene-containing additive (for examples prepared according to the invention) were charged into a five-gallon polyether polyol reactor. The reactor was purged with nitrogen by pressurizing to 40 psia with nitrogen, evacuating to 20 psia and repeating three times. The vacuum valve to the reactor was closed, and the mixture was heated to 100° C. Nitrogen was added to the reactor until a pressure of 20 psia was reached. A propylene oxide (PO) feed into the reactor was initiated. The PO feed rate was controlled via a feedback loop to maintain a total reactor pressure of 45 psia. The amount of PO indicated in Table III (values in grams) as PO-1 was added and the feed was stopped and allowed to cook until the pressure stopped decreasing, indicating the PO was consumed. The time required for the PO addition was recorded. The vacuum valve to the reactor was opened and the reaction mixture was heated under full vacuum to de-water.

De-watering continued at 100° C. until the water level reached 0.40-0.45%, as determined by Karl-Fischer titration. Where necessary, water was added back into the reaction mixture to bring the water content into this range. Sufficient nitrogen was added to bring the reactor pressure to 20 psia, and the second PO feed (PO-2) was initiated. Over the first 120 minutes of the feed, the temperature was increased up to 120° C. in a linear fashion. Again, the PO feed rate was controlled via a feedback loop to maintain 45 psia of pressure during the feed. The time required for the second PO feed was recorded, and the total PO addition time determined by adding the time required for both PO feeds is shown in Table IV. Either sulfuric or lactic acid (see Table IV) was added to neutralize the KOH. For the sulfuric acid neutralized samples, the product was filtered and characterized by viscosity at 25° C., hydroxyl number and appearance (turbid or not). Lactic acid neutralized samples were not filtered prior to characterization.

As can be appreciated by reference to Table IV, the short-chain polyether polyols produced with PEG-300 concentration within the range claimed by the invention (Ex. 12-15) showed a rate acceleration over those produced without any polyoxyethylene-containing compound (Ex. C-9, C-10, C-11). Once again the use of the PEG-300 resulted in a non-turbid sample.

TABLE III

| | Ex. C-9 | Ex. C-10 | Ex. C-11 | Ex. 12 | Ex. 13 | Ex. 14 | Ex. 15 |
|---|---|---|---|---|---|---|---|
| Sucrose | 4,570 | 4,570 | 4,570 | 4,570 | 4,570 | 4,569 | 4,569 |
| Water | 914 | 872 | 828 | 868 | 868 | 868 | 868 |
| 45% aqueous | 124 | 200 | 280 | 120 | 180 | 198 | 285 |
| KOH solution | | | | | | | |
| PEG-300 | — | — | — | 116 | 173 | 539 | 540 |
| PO-1 | 6,635 | 6,635 | 6,635 | 6,000 | 6,000 | 6,635 | 6,635 |
| PO-2 | 6,795 | 6,795 | 6,795 | 7,430 | 7,430 | 6,795 | 6,795 |
| Total PO | 13,430 | 13,430 | 13,430 | 13,430 | 13,430 | 13,430 | 13,430 |

TABLE IV

| | Ex. C-9 | Ex. C-10 | Ex. C-11 | Ex. 12 | Ex. 13 | Ex. 14 | Ex. 15 |
|---|---|---|---|---|---|---|---|
| Additive | None | None | None | PEG-300 | PEG-300 | PEG-300 | PEG-300 |
| (% in product) | | | | (0.64%) | (0.96%) | (3%) | (3%) |
| KOH (%) | 0.3 | 0.5 | 0.7 | 0.3 | 0.45 | 0.5 | 0.7 |
| Total feed (hours) | 17.5 | 12.5 | 9.0 | 13.3 | 7.0 | 7.5 | 8.0 |
| OH number (mg KOH/g) | 390 | 382 | 385 | 375 | 375 | 374 | 377 |
| Viscosity (cP) | 13,420 | 12,023 | 14,476 | 15,100 | 15,900 | 9,000 | 8,692 |
| Neutralization | sulfuric | sulfuric | sulfuric | lactic | lactic | sulfuric | sulfuric |
| | acid | acid | acid | acid | acid | acid | acid |
| Turbidity | no | no | no | no | no | no | no |

The foregoing examples of the present invention are offered for the purpose of illustration and not limitation. It will be apparent to those skilled in the art that the embodiments described herein may be modified or revised in various ways without departing from the spirit and scope of the invention. The scope of the invention is to be measured by the appended claims.

1. A short-chain polyether polyol having a number average molecular weight of less than about 1,200 g/mole and produced by alkoxylating an initiator in the presence of a basic catalyst having at least one cation thereof chelated with about 0.5 wt. % to about 20 wt. % of a polyoxyethylene-containing compound, wherein the weight percentages are based on the weight of the short-chain polyether polyol.

2. The short-chain polyether polyol according to claim 1 having a number average molecular weight of from about 300 g/mole to about 1,000 g/mole.

3. The short-chain polyether polyol according to claim 1 having a number average molecular weight of from about 500 g/mole to about 900 g/mole.

4. The short-chain polyether polyol according to claim 1, wherein the initiator is chosen from C₁-C₃₀ monols, ethylene glycol, diethylene glycol, triethylene glycol, propylene glycol, dipropylene glycol, tripropylene glycol, neopentyl glycol, 1,3 propanediol, 1,4 butanediol, 1,2 butanediol, 1,3 butanediol, 2,3 butanediol, 1,6 hexanediol, water, glycerin, trimethylolpropane, trimethylolethane, ethylene diamine, isomers of toluene diamine, pentaerythritol, α-methylglucoside, sorbitol, mannitol, hydroxymethylglucoside, hydroxypropylglucoside, sucrose, N,N,N′,N′-tetrakis[2-hydroxyethyl or 2-hydroxypropyl]ethylene diamine, 1,4-cyclohexanediol, cyclohexanedimethanol, hydroquinone, resorcinol and mixtures thereof.

5. The short-chain polyether polyol according to claim 1, wherein the basic catalyst is chosen from potassium hydroxide, sodium hydroxide, barium hydroxide and cesium hydroxide.

6. The short-chain polyether polyol according to claim 1, wherein the basic catalyst is potassium hydroxide.

7. The short-chain polyether polyol according to claim 1, wherein the alkylene oxide is chosen from ethylene oxide, propylene oxide, oxetane, 1,2- and 2,3-butylene oxide, isobutylene oxide, epichlorohydrin, cyclohexene oxide, styrene oxide, C₅-C₃₀ α-alkylene oxides and mixtures thereof.

8. The short-chain polyether polyol according to claim 1, wherein the alkylene oxide is propylene oxide.

9. The short-chain polyether polyol according to claim 1, wherein the at least one cation of the basic catalyst is chelated with about 1 wt. % to about 10 wt. % of the polyoxyethylene-containing compound.

10. The short-chain polyether polyol according to claim 1, wherein the at least one cation of the basic catalyst is chelated with about 2 wt. % to about 7 wt. % of the polyoxyethylene-containing compound.

11. A process for producing a short-chain polyether polyol comprising alkoxylating an initiator in the presence of a basic catalyst having at least one cation chelated with about 0.5 wt. % to about 20 wt. % of a polyoxyethylene-containing compound, wherein the short-chain polyether polyol has a number average molecular weight of less than about 1,200 g/mole, wherein the weight percentages are based on the weight of the short-chain polyether polyol.

12. The process according to claim 11, wherein the short-chain polyether polyol has a number average molecular weight of from about 300 g/mole to about 1,000 g/mole.

13. The process according to claim 11, wherein the short-chain polyether polyol has a number average molecular weight of from about 500 g/mole to about 900 g/mole.

14. The process according to claim 11, wherein the initiator is chosen from C₁-C₃₀ monols, ethylene glycol, diethylene glycol, triethylene glycol, propylene glycol, dipropylene glycol, tripropylene glycol, neopentyl glycol, 1,3 propanediol, 1,4 butanediol, 1,2 butanediol, 1,3 butanediol, 2,3 butanediol, 1,6 hexanediol, water, glycerin, trimethylolpropane, trimethylolethane, ethylene diamine, isomers of toluene diamine, pentaerythritol, α-methylglucoside, sorbitol, mannitol, hydroxymethylglucoside, hydroxypropylglucoside, sucrose, N,N,N′,N′-tetrakis[2-hydroxyethyl or 2-hydroxypropyl]ethylene diamine, 1,4-cyclohexanediol, cyclohexanedimethanol, hydroquinone, resorcinol and mixtures thereof.

15. The process according to claim 11, wherein the basic catalyst is chosen from potassium hydroxide, sodium hydroxide, barium hydroxide and cesium hydroxide.

16. The process according to claim 11, wherein the basic catalyst is potassium hydroxide.

17. The process according to claim 11, wherein the alkylene oxide is chosen from ethylene oxide, propylene oxide, oxetane, 1,2- and 2,3-butylene oxide, isobutylene oxide, epichlorohydrin, cyclohexene oxide, styrene oxide, C₅-C₃₀ α-alkylene oxides and mixtures thereof.

18. The process according to claim 11, wherein the alkylene oxide is propylene oxide.

19. The process according to claim 11, wherein the at least one cation of the basic catalyst is chelated with about 1 wt. % to about 10 wt. % of the polyoxyethylene-containing compound.

20. The process according to claim 11, wherein the at least one cation of the basic catalyst is chelated with about 2 wt. % to about 7 wt. % of the polyoxyethylene-containing compound.

21. A rigid polyurethane foam comprising the reaction product of at least one polyisocyanate; and
at least one short chain polyether polyol having a number average molecular weight of less than about 1,200 g/mole and produced by alkoxylating an initiator in the presence of a basic catalyst having at least one cation chelated with about 0.5 wt. % to about 20 wt. % of a polyoxyethylene-containing compound,
optionally in the presence of at least one of blowing agents, surfactants, other cross-linking agents, extending agents, pigments, flame retardants, catalysts and fillers,
wherein the weight percentages are based on the weight of the short-chain polyether polyol.

22. The rigid polyurethane foam according to claim 21, wherein the at least one polyisocyanate is chosen from ethylene diisocyanate, 1,4-tetramethylene diisocyanate, 1,6-hexamethylene diisocyanate, 1,12-dodecane diisocyanate, cyclobutane-1,3-diisocyanate, cyclohexane-1,3- and -1,4-diisocyanate, 1-isocyanato-3,3,5-trimethyl-5-isocyanatomethyl-cyclohexane (isophorone diisocyanate), 2,4- and 2,6-hexahydrotoluene diisocyanate, dicyclohexylmethane-4,4′-diisocyanate (hydrogenated MDI, or HMDI), 1,3- and 1,4-phenylene diisocyanate, 2,4- and 2,6-toluene diisocyanate (TDI), diphenylmethane-2,4′- and/or -4,4′-diisocyanate (MDI), polymeric diphenylmethane diisocyanate (PMDI), naphthylene-1,5-diisocyanate, triphenyl-methane-4,4′,4″-triisocyanate, polyphenyl-polymethylene-polyisocyanates (crude MDI), norbornane diisocyanates, m- and p-isocyanatophenyl sulfonylisocyanates, perchlorinated aryl polyisocyanates, carbodiimide-modified polyisocyanates, urethane-modified polyisocyanates, allophanate-modified polyisocyanates, isocyanurate-modified polyisocyanates, urea-modified polyisocyanates, biuret containing polyisocyanates, isocyanate-terminated prepolymers and mixtures thereof.

23. The rigid polyurethane foam according to claim 21, wherein the at least one polyisocyanate is polymeric diphenylmethane diisocyanate (PMDI).

24. The rigid polyurethane foam according to claim 21, wherein the short-chain polyether polyol has a number average molecular weight of from about 300 g/mole to about 1,000 g/mole.

25. The rigid polyurethane foam according to claim 21, wherein the short-chain polyether polyol has a number average molecular weight of from about 500 g/mole to about 900 g/mole.

26. The rigid polyurethane foam according to claim 21, wherein the initiator is chosen from C₁-C₃₀ monols, ethylene glycol, diethylene glycol, triethylene glycol, propylene glycol, dipropylene glycol, tripropylene glycol, neopentyl glycol, 1,3 propanediol, 1,4 butanediol, 1,2 butanediol, 1,3 butanediol, 2,3 butanediol, 1,6 hexanediol, water, glycerin, trimethylolpropane, trimethylolethane, ethylene diamine, isomers of toluene diamine, pentaerythritol, α-methylglucoside, sorbitol, mannitol, hydroxymethylglucoside, hydroxypropylglucoside, sucrose, N,N,N′,N′-tetrakis[2-hydroxyethyl or 2-hydroxypropyl]ethylene diamine, 1,4-cyclohexanediol, cyclohexanedimethanol, hydroquinone, resorcinol and mixtures thereof.

27. The rigid polyurethane foam according to claim 21, wherein the basic catalyst is chosen from potassium hydroxide, sodium hydroxide, barium hydroxide and cesium hydroxide.

28. The rigid polyurethane foam according to claim 21, wherein the basic catalyst is potassium hydroxide.

29. The rigid polyurethane foam according to claim 21, wherein the alkylene oxide is chosen from ethylene oxide, propylene oxide, oxetane, 1,2- and 2,3-butylene oxide, isobutylene oxide, epichlorohydrin, cyclohexene oxide, styrene oxide, C₅-C₃₀ α-alkylene oxides and mixtures thereof.

30. The rigid polyurethane foam according to claim 21, wherein the alkylene oxide is propylene oxide.

31. The rigid polyurethane foam according to claim 21, wherein the at least one cation of the basic catalyst is chelated with about 1 wt. % to about 10 wt. % of the polyoxyethylene-containing compound.

32. The rigid polyurethane foam according to claim 21, wherein the at least one cation of the basic catalyst is chelated with about 2 wt. % to about 7 wt. % of the polyoxyethylene-containing compound.

33. A process for producing a rigid polyurethane foam comprising reacting
at least one polyisocyanate; and
at least one short chain polyether polyol having a number average molecular weight of less than about 1,200 g/mole and produced by alkoxylating an initiator in the presence of a basic catalyst having at least one cation chelated with about 0.5 wt. % to about 20 wt. % of a polyoxyethylene-containing compound,
optionally in the presence of at least one of blowing agents, surfactants, other cross-linking agents, extending agents, pigments, flame retardants, catalysts and fillers,
wherein the weight percentages are based on the weight of the short-chain polyether polyol.

34. The process according to claim 33, wherein the at least one polyisocyanate is chosen from ethylene diisocyanate, 1,4-tetramethylene diisocyanate, 1,6-hexamethylene diisocyanate, 1,12-dodecane diisocyanate, cyclobutane-1,3-diisocyanate, cyclohexane-1,3- and -1,4-diisocyanate, 1-isocyanato-3,3,5-trimethyl-5-isocyanatomethyl-cyclohexane (isophorone diisocyanate), 2,4- and 2,6-hexahydrotoluene diisocyanate, dicyclohexylmethane-4,4′-diisocyanate (hydrogenated MDI, or HMDI), 1,3- and 1,4-phenylene diisocyanate, 2,4- and 2,6-toluene diisocyanate (TDI), diphenylmethane-2,4′- and/or -4,4′-diisocyanate (MDI), polymeric diphenylmethane diisocyanate (PMDI), naphthylene-1,5-diisocyanate, triphenyl-methane-4,4′,4″-triisocyanate, polyphenyl-polymethylene-polyisocyanates (crude MDI), norbornane diisocyanates, m- and p-isocyanatophenyl sulfonylisocyanates, perchlorinated aryl polyisocyanates, carbodiimide-modified polyisocyanates, urethane-modified polyisocyanates, allophanate-modified polyisocyanates, isocyanurate-modified polyisocyanates, urea-modified polyisocyanates, biuret containing polyisocyanates, isocyanate-terminated prepolymers and mixtures thereof.

35. The process according to claim 33, wherein the at least one polyisocyanate is polymeric diphenylmethane diisocyanate (PMDI).

36. The process according to claim 33, wherein the short-chain polyether polyol has a number average molecular weight of from about 300 g/mole to about 1,000 g/mole.

37. The process according to claim 33, wherein the short-chain polyether polyol has a number average molecular weight of from about 500 g/mole to about 900 g/mole.

38. The process according to claim 33, wherein the initiator is chosen from C₁-C₃₀ monols, ethylene glycol, diethylene glycol, triethylene glycol, propylene glycol, dipropylene glycol, tripropylene glycol, neopentyl glycol, 1,3 propanediol, 1,4 butanediol, 1,2 butanediol, 1,3 butanediol, 2,3 butanediol, 1,6 hexanediol, water, glycerin, trimethylolpropane, trimethylolethane, ethylene diamine, isomers of toluene diamine, pentaerythritol, α-methylglucoside, sorbitol, mannitol, hydroxymethylglucoside, hydroxypropylglucoside, sucrose, N,N,N′,N′-tetrakis[2-hydroxyethyl or 2-hydroxypropyl]ethylene diamine, 1,4-cyclohexanediol, cyclohexanedimethanol, hydroquinone, resorcinol and mixtures thereof.

39. The process according to claim 33, wherein the basic catalyst is chosen from potassium hydroxide, sodium hydroxide, barium hydroxide and cesium hydroxide.

40. The process according to claim 33, wherein the basic catalyst is potassium hydroxide.

41. The process according to claim 33, wherein the alkylene oxide is chosen from ethylene oxide, propylene oxide, oxetane, 1,2- and 2,3-butylene oxide, isobutylene oxide, epichlorohydrin, cyclohexene oxide, styrene oxide, C₅-C₃₀ α-alkylene oxides and mixtures thereof.

42. The process according to claim 33, wherein the alkylene oxide is propylene oxide.

43. The process according to claim 33, wherein the at least one cation of the basic catalyst is chelated with about 1 wt. % to about 10 wt. % of the polyoxyethylene-containing compound.

44. The process according to claim 33, wherein the at least one cation of the basic catalyst is chelated with about 2 wt. % to about 7 wt. % of the polyoxyethylene-containing compound.
